(19) United States
(12) Patent Application Publication
(10) Pub. No.: US 2002/0102589 A1
(43) Pub. Date: Aug. 1, 2002

(54) Microarrays and methods for evaluating activity of compounds having estrogen-like activity

(76) Inventors: Ryoiti Kiyama, Ibaraki (JP); Shinobu Oguchi, Tokyo (JP)

Correspondence Address: Stephen B. Maebius, FOLEY & LARDNER, Suite 500, 3000 K Street, N.W., Washington, DC, 20007-5109, US

(21) Appl. No.: 09/975,316

(22) Filed: Oct. 12, 2001

Related U.S. Application Data
(60) Provisional application No. 60/300,835, filed on Jun. 27, 2001.

(30) Foreign Application Priority Data
Oct. 13, 2000 (JP) 2000-313403

(51) Int. Cl. C12Q 1/68; G06F 19/00

(52) U.S. Cl. 435/6; 702/20

(57) ABSTRACT

In order to detect the effects of environmental hormones, the effect of chemical substances having estrogen-like activity are detected and evaluated

This invention is characterized in that DNA fragments containing portions or wholes of genes and/or ESTs (Expressed Sequence Tags) whose expression is affected by chemical substances having estrogen-like activity are immobilized on the basal plate of the microarry.

### FIELD OF THE INVENTION

This invention relates to a microarray comprising DNA probes immobilized on the basal plate thereof and a method for evaluating activity of compounds having estrogen-like activity that uses this microarray.

### BACKGROUND OF THE INVENTION

So far, the so-called environmental hormones are known to be chemical substances such as those that have estrogen-like activity as natural estrogens, 17β-estradiol for example, do. These environmental hormones are endocrine disrupting chemicals towards the human body and have greatly deleterious effects on each of the tissues within the human body. Therefore, precise evaluation of the activity of environmental hormones, together with detection of environmental hormones, in each of the tissues of the human body is desired. Although detection of environmental hormones by sampling each of the tissues of the human body has been possible, detection of the activity of environmental hormones in each of the tissues has not been possible.

Microarrays that are used for monitoring gene expression, determination and variant analysis of nucleotide sequence, and such are known. A microarray is also called a DNA chip and comprises a basal plate made of slide glass, silicone, or the like, and DNA fragments immobilized as an array on this basal plate. With this microarray, DNAs contained in a sample can be detected by hybridizing them with the DNA fragments immobilized on the basal plate. Since the DNA within the sample is radiolabeled or fluorescently labeled, detection with radio imaging scanner, fluorescence imaging scanner, or the like is possible.

In this way, microarrays enable quantitatively detecting DNA contained within a sample and monitoring gene expression and the like. However, methods for evaluating the detection and the effect of the above-mentioned estrogen using microarrays are not established.

### SUMMARY OF THE INVENTION

This invention has been made in view of the current state of affairs as mentioned above, and an objective of this invention is to provide microarrays capable of detecting and evaluating the effect of chemical substances having estrogen-like activity and methods for evaluating activity of compounds having estrogen-like activity, for the purpose of detecting the effect of environmental hormones.

The microarray of this invention, which has accomplished the above-mentioned objective, is characterized in that DNA fragments containing portions or the wholes of genes and/or ESTs (Expressed Sequence Tags) whose expression is affected by chemical substances having estrogen-like activity are immobilized on the basal plate of the microarray.

In this invention, it is preferable that the genes and/or the ESTs are selected from the group consisting of those whose expression level changes in breast cancer-derived MCF-7 cells in the presence of estrogen.

Furthermore, it is preferable that the above-mentioned genes and/or ESTs belong to any one of groups (1), (2), and (3) below:

group (1): genes and/or ESTs whose expression level increases owing to chemical substances having estrogen-like activity,

group (2): genes and/or ESTs whose expression level decreases owing to chemical substances having estrogen-like activity, and

group (3): genes and/or ESTs whose expression level changes smaller and whose expression level is higher, compared to the groups (1) and (2) above.

Also, it is preferable that, in the microarray, at least one or more genes and/or ESTs selected from the group consisting of genes and/or ESTs belonging to the group (1) above, genes and/or ESTs belonging to the group (2) above, and genes and/or ESTs belonging to the group (3) above are immobilized.

Furthermore, it is preferable that, in the microarray of this invention, genes and/or ESTs constitutively expressing regardless of the presence or absence of chemical substances having estrogen-like activity are further immobilized on the basal plate of the microarray.

On the other hand, in the microarray of this invention, it is preferable that the above-mentioned genes and/or ESTs belong to any one of groups (4), (5), (6), (7), and (8) below:

group (4): genes and/or ESTs related to cancer,

group (5): genes and/or ESTs involved in cell proliferation and/or division,

group (6): genes and/or ESTs encoding polypeptides functioning as receptors,

group (7): genes and/or ESTs involved in transcription reaction, and

group (8): genes and/or ESTs involved in cell structure.

Here, it is preferable that at least one or more genes and/or ESTs selected from the group consisting of genes and/or ESTs belonging to the group (4) above, genes and/or ESTs belonging to the group (5) above, genes and/or ESTs belonging to the group (6) above, genes and/or ESTs belonging to the group (7) above, and genes and/or ESTs belonging to the group (8) above are immobilized.

Also, it is preferable that, in the microarray of this invention, the above-mentioned genes and/or ESTs belong to any one of groups (9), (10), and (11) below:

group (9): genes and/or ESTs functioning in tissues on which estrogen acts,

group (10): genes and/or ESTs characteristic of epithelial cells, and

group (11): genes and/or ESTs functioning in nerve cells.

Here, it is preferable that at least one or more genes and/or ESTs selected from the group consisting of genes and/or ESTs belonging to the group (9) above, genes and/or ESTs belonging to the group (10) above, and genes and/or ESTs belonging to the group (11) above are immobilized.

Furthermore, it is preferable that, in the microarray of this invention, the above-mentioned genes and/or ESTs belong to group (12) or (13) below:

group (12): genes and/or ESTs whose expression level increases owing to estrogen in cells resistant to antagonists of estrogen, and

group (13): genes and/or ESTs whose expression level decreases owing to estrogen in cells resistant to antagonists of estrogen.

Here, it is preferable that at least one or more genes and/or ESTs selected from the group consisting of genes and/or ESTs belonging to the group (12) above and genes and/or ESTs belonging to the group (13) above are immobilized.

On the other hand, the method of this invention for evaluating activity of compounds having estrogen-like activity comprises utilizing the microarray of any one of claims 1 to 11.

Furthermore, this invention relates to a method for evaluating activity of compounds having estrogen-like activity, the method comprising the steps of:

(a) preparing a nucleic acid sample from a cell with which an effect of a compound having estrogen-like activity is to be evaluated,

(b) contacting the nucleic acid sample with the microarray of any one of claims 1 to 11,

(c) detecting a nucleic acid hybridizing with the microarray, and

(d) comparing a result of the detecting of (c) to a result detected using a nucleic acid sample prepared from a control cell.

Here, it is preferable that the control cell is an MCF-7 cell treated with 17β-estradiol.

### DETAILED DESCRIPTION OF THE INVENTION

The microarrays and the methods of this invention for evaluating activity of compounds having estrogen-like activity are described in detail below.

The microarray to which this invention is applied has multiple DNA fragments immobilized on the basal plate of the microarray. Here, DNA fragments contain a portion or the whole of genes and/or ESTs whose expression is affected by chemical substances having estrogen-like activity. Specifically, examples of the genes and ESTs include those indicating responsiveness towards 17β-estradiol in a breast cancer-derived MCF-7 cell line or MCF-7 cell line-derived cell line. That is, in a breast cancer-derived MCF-7 cell line or MCF-7 cell line-derived cell line, it is possible to use DNA fragments containing a portion or the whole of the genes or ESTs whose expression level changes in response to the presence or absence of 17β-estradiol.

Herein, “expression level changes in response to the presence or absence of 17β-estradiol” means that either the increase in the amount of mRNA is twofold or more when 17β-estradiol is present, or the decrease in the amount of mRNA is ½ or lower when 17β-estradiol is present.

Examples of genes or ESTs indicating responsiveness towards 17β-estradiol in a breast cancer-derived MCF-7 cell line or MCF-7 cell line-derived cell line include

group (1): those whose expression level increases owing to 17β-estradiol,

group (2): those whose expression level decreases owing to 17β-estradiol, and

group (3): those whose expression level changes smaller and whose expression level is higher, compared to (1) and (2) above.

Herein, the group (1) means those whose expression level increases to threefold or more owing to the presence of 17β-estradiol, and the group (2) means those whose expression level decreases to ⅓ or lower owing to the presence of 17β-estradiol. In addition, the group (3) means those whose expression level increases to more than twofold and less than threefold or decreases to more than ⅓ and less than ½ owing to the presence of 17β-estradiol and whose expression level is the same as or not less than the expression level of keratin 6B, which is one of the genes used as a control.

More specifically, examples of genes or ESTs of the group (1) include

asparagine synthetase {L35946};

trefoil factor 1 {X05030};

ESTs {N35555};

early growth response 3 {X63741};

amphiregulin {M30704};

matrix Gla protein {AA484893};

tumor protein D52-like 1 {U44427};

phosphoenolpyruvate carboxykinase 2 {X92720};

KIAA1051 {AB028974};

EST {AA587912};

transcobalamin I {J05068};

tryptophanyl-tRNA synthetase {X62570};

stanniocalcin 2 {AB012664};

solute carrier family 1 {L14595};

solute carrier family 7 {M80244};

antigen identified by monoclonal antibodies 4F2, TRA1. 10, TROP4, and T43 {AB018010};

calgranulin B {X06233};

CCAAT/enhancer binding protein (C/EBP), beta {AA557306};

FOS-like antigen 2 {AI088306};

prostate differentiation factor {AA216685};

heat shock 70-kD protein 5 {AI878886};

activating transcription factor 3 {N39944};

S100 calcium-binding protein P {AI151190};

motilin {X15393};

phorbol-12-myristate-13-acetate-induced protein 1 {D90070};

down-regulated in adenoma {L02785};

interferon stimulated genet {X89773};

glutamic-oxaloacetic transaminase 1 {M37400};

methylene tetrahydrofolate dehydrogenase (NAD⁺ dependent) {X16396};

retinoblastoma-binding protein 8 {U72066};

microtubule-associated protein 1B {L06237};

EST {AI185199};

phosphoinositide-3-kinase, class 3 {Z46973};

EST {AA975298};

aldo-keto reductase family 1, member C4 {T73188};

semaphorin {AI683760};

unc-51-like kinase 1 {AB018265};

insulin-like growth factor-binding protein 4 {M62403};

hyaluronan-mediated motility receptor (RHAMM) {U29343};

alanyl-tRNA synthetase {D32050};

PDZ domain containing 1 {AF012281};

cyclin A1 {U66838};

TGFB inducible early growth response {AF050110};

tumor rejection antigen (gp96) 1 {X15187};

properdin {X72875};

EST {AA629308};

glutamine-fructose-6-phosphate transaminase 1 {M90516};

neuropeptide Y receptor Y1 {M84755};

EST {U82984};

EST {AA682502};

ribosomal protein S6 kinase, 90kD, polypeptide 3 {U08316};

glycyl-tRNA synthetase {D30658};

FOS-like antigen 2 {X16706};

DKFZP566G223 protein {AI261366};

EST {AI880413};

EST {X62585};

v-jun avian sarcoma virus 17 oncogene homolog {AI598150};

nuclear receptor interacting protein 1 {X84373}; and

exportin {AF039022}.

GenBank accession numbers are indicated in parentheses.

Examples of genes or ESTs of the group (2) include

bassoon {Y18448};

KIAA0018 {D13643};

estrogen receptor {X03635};

Rho GDP dissociation inhibitor (GDI) alpha {X69550};

absent in melanoma 1 {U83115};

fucosyltransferase 8 {Y17977};

catenin {U96136};

enolase 3 {X56832};

clusterin {X14723};

paired basic amino acid cleaving system 4 {D87993};

KIAA0307 {AB002305};

heat shock 70-kD protein 1 {M59828};

cadherin 18 {U59325};

isocitrate dehydrogenase 2 {X69433};

tetraspan 1 {AF065388};

quiescin Q6 {U97276};

solute carrier family 12 {U30246};

selenium binding protein 1 {U29091};

EGF-containing fibulin-like extracellular matrix protein 1 {U03877};

insulin-like growth factor binding protein 5 {AA374325}; and

Human insulin-like growth factorbindingprotein 5 (IGFBP5) {L27560}.

Examples of genes or ESTs of the group (3) include

argininosuccinate synthetase {AI660571};

ferritin, heavy polypeptide 1 {AA102267};

tyrosyl-tRNA synthetase {U89436};

H3 histone, family 3B (H3.3B) {Z48950};

alpha-1-antichymotrypsin {X00947};

SH3-domain binding protein 5 {AB005047};

pituitary tumor-transforming 1 {AA430241};

cathepsin D {M63138};

KIAA0587 {AB011159};

solute carrier family 1, member 5 {AF105826};

EST {AI949781};

corneodesmosin {L20815};

EST {AI188401};

keratin 8 {Incyte PD: 4337383} {X74929};

serine hydroxymethyltransferase 2 {U23143};

EST {AI332415};

EST {AI492976};

inhibitor of DNA binding 4 {AL022726};

mannosidase, alpha, class 1A, member 1 {X74837};

TATA box binding protein (TBP)-associated factor, RNA polymerase II, G {U21858};

CDC6 homolog {AF022109};

EST {AI767533};

histone deacetylase 6 {AJ011972};

HS1 binding protein {N28312};

isoleucine-tRNA synthetase {D28473};

KIAA0551 {AB011123};

poliovirus receptor {X64116 };

protein geranylgeranyltransferase type I, beta subunit {AA481712};

reticulocalbin 1 {D42073};

stearoyl-CoA desaturase {AA143530};

thrombospondin 3 {AI679881 };

CDP-diacylglycerol-inositol 3-phosphatidyltransferase {AF014807};

Homo sapiens clone 23783 {AF054996};

KIAA0123 {D21064};

mal {X76220};

40-kD glycoprotein, identified by monoclonal antibody GA733 {X13425};

protein tyrosine phosphatase, non-receptor type 18 {X79568};

U5 snRNP-specific protein {D21163};

chromobox homolog 1 {AL046741};

Human clone 23855 {U79302};

KIAA0058 {AL036958};

ras homolog gene family, member C {L25081};

eukaryotic translation initiation factor 3, subunit 9 {U78525};

granulin {AF055008};

KIAA0196 {D83780};

protein kinase C substrate 80K-H {J03075};

sorbitol dehydrogenase {U07361};

aconitase 2 {U87939};

synaptogyrin 2 {AJ002308};

carnitine palmitoyltransferase I {L39211};

fructose-bisphosphatase 1 {M19922};

GATA-binding protein 3 {X55122};

Homo sapiens DNA from chromosome 19, cosmid F21856 {AC004030};

MYC promoter-binding protein 1 {AA482422};

enolase 2, (gamma, neuronal) {X51956};

calpain {AC002984};

general transcription factor II, i, pseudogene 1 {AI700706}; and

protein kinase C, delta {D10495}

Furthermore, in this invention, the genes and/or ESTs to be immobilized on the basal plate are not limited to those belonging to the above-mentioned groups (1), (2), and (3), and any genes and/or any ESTs may be used as long as their expression is affected by chemical substances having estrogen-like activity. The term “chemical substances having estrogen-like activity” used herein means a chemical substance that affects female reproductive organs and such similarly to a human sex hormone, estrogen (a general term for hormones that are steroidal chemical substances secreted from ovarian follicles, placenta, and such and that induce the development of female reproductive organs such as follicles and mammary glands or other organs (such as brain)). Although the functional mechanism of estrogen itself is not yet completely understood, estrogen is thought to affect the expression of other genes by binding to estrogen receptors. Therefore, estrogen is thought to affect tissues such as brain, liver, muscle, bone cells, stomach, and so on where estrogen receptor gene is expressed.

Examples of chemical substances having estrogen-like activity include natural or synthesized female hormones such as 17β-estradiol, estrone, estriol, diethylstilbestrol, and such. In addition, examples of chemical substances having estrogen-like activity include plant estrogens such as coumestrol and daidzein. Furthermore, examples of chemical substances having estrogen-like activity include agricultural chemicals such as DDT, aldrin, and heptachlor. Furthermore, examples of chemical substances having estrogen-like activity include industrial raw materials, materials, products, and by-products such as dioxin, PCB, nonyphenol, bisphenol A, phthalic acid esters, and tributyltin compounds.

The DNA fragment to be immobilized on the basal plate may contain the whole or a portion of the genes and/or the ESTs described above. The term “a portion of a gene and/or an EST” used herein means a portion of the gene and/or the EST described above and a nucleotide sequence equivalent to at least 300 to 500 base pairs.

In addition, it is preferable that, in a microarray, genes and/or ESTs constitutively expressing regardless of the presence or absence of chemical substances having estrogen-like activity (hereinafter, referred to as control genes and the like) are immobilized on the basal plates. For the control genes and the like, it is preferable to select several of those that have differences in their expression levels. Genes constitutively expressing regardless of the presence of absence of chemical substances having estrogen-like activity and having differences in their expression levels are, for example,

keratin 6B {L42611};

actin, beta {X00351};

ribosomal protein L35 {AI815757};

solute carrier family 5, member 1 {Z80998};

zinc finger protein 147 {D21205};

general transcription factor IIH, polypeptide 2 {AI239815};

glutamate-cysteine ligase {L35546};

nucleophosmin {AA173870};

solute carrier family 29, member 2 {AI342303}; and

tumor necrosis factor receptor superfamily, member 7 {AA994925}.

The expression level of the genes and/or ESTs of the groups (1) (2), and (3) can be corrected by immobilizing control genes and such on the basal plate. That is, by correcting so that the expression level of the control genes and such may be always constant, the expression level of the genes and/or ESTs of the groups (1), (2), and (3) can be corrected. Thus, the changes in the expression level of genes and/or ESTs of the groups (1), (2), and (3) can be detected with certainty.

The expression level of genes and/or ESTs indicated in the groups (1), (2), and (3) may be corrected with accuracy by choosing several of control genes and such that have different expression levels. That is, when the expression level of a particular gene and/or EST is to be corrected, correction can be carried out with accuracy using control genes and such that indicate expression levels close to the expression levels of the genes and/or ESTs in question.

When the DNA fragments and control genes are immobilized on the basal plate, a conventionally known technique can be used. For example, the surface of the basal plate can be treated with polycations such as polylysines to electrostatically bind the DNA fragments through their charges on the surface of the basal plate, and techniques to covalently bind the 5′-end of the DNA fragments to the basal plate may be used. Also, a basal plate that has linkers on its surface can be produced, and functional groups that can form covalent bonds with the linkers can be introduced at the end of the DNA fragments. Then, by forming a covalent bond between the linker and the functional group, the DNA fragments and such can be immobilized.

The expression levels of the genes and/or ESTs indicating responsiveness towards estrogen in the cell to be evaluated can be measured by using a microarray composed as described above. Measuring the expression level of genes and/or ESTs in the cell to be evaluated enables evaluation of the effects of chemical substances having estrogen-like activity, such as environmental hormones, etc.

In this invention, there are no particular limitations on cells to be evaluated. Cells obtained from human tissues such as mammary glands, uterus, liver, brain, stomach, and such may be used as cells to be evaluated. To evaluate the effect of environmental hormones and such in the cells to be evaluated, first, total mRNA is extracted from the cells. When mRNA is extracted, a conventionally known technique may be used, and a commercially available mRNA purification kit (for example, MACS ; Miltenyi Biotec) may be used.

Next, with the extracted mRNA as a template, cDNAs are produced using a reverse transcriptase (for example, SuperScript Reverse Transcriptase ; GibcoBRL) and labeled dNTP (for example, Cy3-dUTP and Cy5-dUTP ; Amersham Pharmacia Biotech), and a cDNA sample that reflects the amount of genes expressed within the cells to be evaluated is prepared. This causes labeled cDNA to be included in the cDNA sample. Here, either fluorescent label or radiolabel may be used as a label.

The cDNA sample prepared in this manner is applied to the above-mentioned microarray in its single stranded denatured form, and cDNAs included in the cDNA sample are hybridized with the genes and/or ESTs immobilized on the basal plate. Here, hybridization with cDNA can be accomplished, for example, by incubating at 65° C. for 10 to 20 hours.

Then, cDNAs that hybridized with the DNA fragments on the microarray are detected. In cases where the hybridized cDNAs are fluorescently labeled, the fluorescence is detected with, for example, a fluorescence laser microscope and a CCD camera, and the fluorescence intensity is analyzed with a computer. Similarly, in cases where the hybridized cDNAs are radiolabeled, detection can be carried out using an RI image scanner and such, and the intensity of the radiation can be analyzed with a computer.

The effect of chemical substances having estrogen-like activity on the cells to be evaluated can be determined using a microarray by detecting the expression level of the above-mentioned genes and/or ESTs in the cells to be evaluated. In other words, using the above-mentioned microarray to measure the expression of genes and ESTs in these cells, the effects of environmental hormones and such may be evaluated.

This evaluation uses an evaluation value that expresses the sum of the expression levels of genes and/or ESTs contained in the DNA fragments immobilized on the basal plate, as a relative value to the sum of the expression levels of genes and/or ESTs in MCF-7 cells in the presence of 10 nM of 17β-estradiol. That is, the evaluation value is calculated as a relative value when the total expression level of genes and/or ESTs in MCF-7 cells in the presence of 10 nM17β-estradiol is taken to be equal to one.

The calculation procedure for the evaluation value is described below. Specifically, when microarrays with immobilized DNA fragments containing the 138 kinds of genes and ESTs indicated in the above-mentioned groups (1), (2), and (3) are used, first, the detected fluorescence intensity is converted to a numerical value to calculate the expression level, En (n=1 to 138), of genes and such that hybridize with the DNA fragments immobilized on the basal plate. Based on the expression level of the control genes and such, this En value is converted to a corrected value. That is, by correcting based on the expression level of the above-mentioned control genes and such, En can be calculated as a relative value in multiple microarrays.

Next, when the expression level of the 138 kinds of genes and ESTs indicated in the groups (1), (2), and (3) in MCF-7 cells on which 10 nM 17β-estradiol acts is denoted as BDEn (n=1 to 138), a coefficient t satisfying

Σ|BDEn|=t(Σ|En|)

is calculated. Then, t is multiplied by the expression level, En (n=1 to 138), of the hybridized genes and such to calculate the corrected value for gene expression level, BDE'n (n=1 to 138). That is, the corrected values for the expression level for the 138 kinds of genes and ESTs, BDE'n (n=1 to 138), are calculated according to

BDE'n=tEn (n=1 to 138).

Next, for BDE'n, a relative value to BDEn is calculated, and the sum of the absolute value of this relative value is calculated as C. That is, according to

C=Σ|BDE'n/BDEn| (n=1 to 138),

C is calculated.

The evaluation value can then be calculated with respect to this C value, as a relative value to the expression level when MCF-7 cells are used (Σ|BDEn/BDEn|=138, (n=1 to 138)). That is, according to

(evaluation value)=C/138

the evaluation value is calculated. Since this evaluation value becomes a relative value when the expression level of genes and ESTs in MCF-7 cells in the presence of 10 nM 17β-estradiol is taken to be 1.00, it will reflect the effect of chemical substances having estrogen-like activity in the cells to be evaluated.

In addition, the evaluation value can be calculated at this point by considering the weighting factor based on the characteristics and such of genes and ESTs for which the expression levels are measured by DNA fragments immobilized on the basal plate. That is, for each of the 138 kinds of genes and ESTs, a weighting factor Kn (n=1 to 138) can be set in advance to calculate the evaluation value according to

(evaluation value)=C/138

=(ΣKn|BDE'n/BDEn|)/(ΣKn|BDEn/BDEn|)

=(ΣKn|BDE'n/BDEn|)/(ΣKn)

where (n=1 to 138).

Calculation of the evaluation value in this manner, considering the weighting factors for the genes and ESTs, makes the resulting evaluation value clearly reflect the effect of chemical substances having estrogen-like activity in the cells to be evaluated. For the control genes and such, the weighting factor is taken to be zero in the calculation.

Here, weighting factor Kn is not set in a restrictive manner according to genes and/or ESTs but rather, it is preferable that this value is changed, considering the current state of the evaluation results, in order to obtain a more accurate evaluation result. Especially, the value of weighting factor Kn is preferably set to various values depending on the tissue from which the cells to be evaluated are derived, and on the compound having estrogen-like activity. Accordingly, this evaluation method enables a highly accurate evaluation tailored to an objective.

In addition, by using the cells obtained from human tissues such as mammary glands, uterus, liver, brain, and stomach as evaluation subjects, this method can distinguish the effect of chemical substances having estrogen-like activity on these tissues. That is, this microarray enables evaluation of the effect of chemical substances having estrogen-like activity on the tissues.

In addition, this microarray not only allows evaluation of the effect of chemical substances having estrogen-like activity on the tissues as described above, but it can also elucidate the estrogen activity of known or unknown chemical substances. That is, by using this microarray for chemical substances whose effects on cells are unknown, one can evaluate whether or not these substances have estrogen-like activity.

Furthermore, this microarray allows distinguishment of whether an activity is enhanced or inhibited when a chemical substance having estrogen-like activity coexists with another chemical substance.

The microarray of this invention not only evaluates the effect of chemical substances having estrogen-like activity on cells to be evaluated, as described above, but can also indicate the details of this effect. Especially, in the above-mentioned microarray, the 138 kinds of genes and ESTs of the groups (1), (2), and (3) can be categorized into

group (4): genes and/or ESTs related to cancer,

group (5): genes and/or ESTs involved in cell proliferation and/or division,

group (6): genes and/or ESTs encoding polypeptides functioning as receptors,

group (7): genes and/or ESTs involved in transcription reaction, and

group (8): genes and/or ESTs involved in cell structure.

Specifically, examples of genes and/or ESTs of the group (4) related to cancer (expression genes or cancer suppressor genes) include

trefoil factor 1 {X05030};

tumor protein D52-like 1 {U44427};

phorbol-12-myristate-13-acetate-induced protein 1 {D90070};

down-regulated in adenoma {L02785};

retinoblastoma-binding protein 8 {U72066};

tumor rejection antigen (gp96) 1 {X15187};

v-jun avian sarcoma virus 17 oncogene homolog {AI598150};

pituitary tumor-transforming 1 {AA430241};

ras homolog gene family, member C {L25081};

MYC promoter-binding protein 1 {AA482422}; and

absent in melanoma 1 {U83115}.

Examples of genes and/or ESTs the group (5) related to cell proliferation and/or division include

early growth response 3 {X63741};

amphiregulin {M30704};

insulin-like growth factor-binding protein 4 {M62403};

cyclin A1 {U66838};

TGFB inducible early growth response {AF050110};

CDC6 homolog {AF022109};

catenin, delta 2 {U96136};

cadherin 18 {U59325};

EGF-containing fibulin-like extracellular matrix protein 1 {U03877};

insulin-like growth factor binding protein 5 {AA374325};

Human insulin-like growth factorbindingprotein5 (IGFBP5) {L27560};

cyclin A1 {U66838}; and

ribosomal protein S6 kinase, 90kD, polypeptide 3 {U08316}.

Examples of genes and/or ESTs of the group (6) encoding polypeptides functioning as receptors include

hyaluronan-mediated motility receptor (RHAMM) {U29343};

neuropeptide Y receptor Y1 {M84755};

nuclear receptor interacting protein 1 {X84373};

poliovirus receptor {X64116}; and

estrogen receptor 1 {X03635}.

Examples of genes and/or ESTs of the group (7) involved in transcription reaction include

CCAAT/enhancer binding protein (C/EBP), beta {AA557306};

FOS-like antigen 2 {AI088306};

prostate differentiation factor {AA216685};

activating transcription factor 3 {N39944};

FOS-like antigen 2 {X16706};

H3 histone, family 3B (H3.3B) {Z48950};

TATA box binding protein (TBP)-associated factor, RNA polymerase II, G {U21858};

histone deacetylase 6 {AJ011972};

GATA-binding protein 3 {X55122};

general transcription factor II, i, pseudogene 1 {AI700706};

heat shock 70-kD protein 1 {AI700706}.

Examples of genes and/or ESTs of the group (8) involved in cell structure include

H3 histone, family 3B {Z48950}; and

histone deacetylase 6 {AJ011972}.

By individually evaluating the expression level of genes and/or ESTs categorized into the groups (4) to (8) in this manner, it is possible to distinguish how chemical substances having estrogen-like activity affect the cells to be evaluated. In other words, it is preferable that, in the microarrays, the DNA fragments containing a portion or the whole of genes belonging to the groups (4) to (8) are immobilized. In this case, the use of microarrays allows distinction of the types of effects of chemical substances having estrogen-like activity on the cells to be evaluated.

Additionally, the 138 kinds of genes and ESTs of the groups (1), (2), and (3) can be categorized into groups of genes that show tissue-specific expression or function. That is, the 138 kinds of genes and ESTs can be categorized into

group (9): genes and/or ESTs functioning in tissues on which estrogen acts,

group (10): genes and/or ESTs characteristic of epithelial cells, and

group (11): genes and/or ESTs functioning in nerve cells.

Specifically, examples of genes and/or ESTs of the group (9) functioning in tissues on which estrogen acts include

trefoil factor 1 {X05030}; and

estrogen receptor 1 {X03635}.

Examples of genes and/or ESTs of the group (10) characteristic of epithelial cells include

tumor protein D52-like 1 {U44427};

solute carrier family 1, member 4 {L14595};

solute carrier family 12, member 2 {U30246}; and

EGF-containing fibulin-like extracellular matrix protein 1 {U03877}.

Examples of genes and/or ESTs of the group (11) functioning in nerve cells include

amphiregulin {M30704};

neuropeptide Y receptor Y1 {M84755};

synaptogyrin 2 {AJ002308}; and

protein tyrosine phosphatase, non-receptor type 18 {X79568}.

By evaluating the expression level of genes categorized into the groups (9) to (11) in this manner, the tissues on which the chemical substances having estrogen-like activity has an effect can be distinguished in the cells to be evaluated.

Furthermore, the 138 kinds of genes and ESTs of the groups (1) (2), and (3) may be categorized according to the responsiveness towards tamoxifen, which is an antagonist of estrogen. That is, these 138 kinds of genes and ESTs may be classified into

group (12): genes and/or ESTs whose expression level increases owing to estrogen in tamoxifen-resistant cells, and

group (13): genes and/or ESTs whose expression level decreases owing to estrogen in tamoxifen-resistant cells.

Specifically, examples genes and/or ESTs of the group (12) whose expression level increases owing to estrogen in tamoxifen-resistant cells include

down-regulated in adenoma (DRA) {AI700706}; and

v-jun avian sarcoma virus 17 oncogene homolog {AI598150}.

Examples of genes and/or ESTs of the group (13) whose expression level decreases owing to estrogen in tamoxifen-resistant cells include

carnitine palmitoyltransferase I {L39211};

cadherin 18 {U59325};

insulin-like growth factor binding protein 5 {AA374325}; and

Human insulin-like growth factor binding protein 5 {L27560}.

By evaluating the expression levels of genes classified into the groups (12) and (13) in this manner, the effect of chemical substances having estrogen-like activity can be distinguished using tamoxifen-resistant cells as the targets of evaluation.

As described in detail above, this invention provides a microarray that can distinguish the effect of chemical substances having estrogen-like activity on cells to be evaluated. Using this microarray, it is possible to evaluate the effect of environmental hormones and such on cells to be evaluated.

In addition, according to this invention, measurement of the expression levels of genes and/or ESTs in the cells to be evaluated may provide a method for evaluating activity of compounds having estrogen-like activity. The use of this method for evaluating activity of compounds having estrogen-like activity enables evaluation of the effect of environmental hormones and such on the cells to be evaluated.

All the references cited herein are incorporated by reference.

Hereinafter, the use of the microarray of this invention to evaluate the effect of 17β-estradiol in the MCF-7 cell derived tamoxifen resistant strain clone 9 cells will be described as examples. However, this invention is not to be construed as being limited thereto.

### EXAMPLE

(1) Preparation of cDNA sample

In this example, the mRNA extracted from MCF-7 cell-derived tamoxifen-resistant strain clone 9 cells using MACS mRNA separation/purification kit (Miltenyi Biotec) was used as a template to synthesize the labeled cDNA sample.

The labeling reaction was carried out as described below. First, 2 μg of poly(A) RNA and 4.5 μg of oligo(dT) primer were mixed. This was then denatured at 70° C. for 10 minutes and cooled rapidly. Immediately, 6 μl of 5X Superscript buffer (GibcoBRL), 3 μl of DTT, 0.6 μl of 50X dNTP (Promega), 3 μl of Cy3-dUTP or Cy5-dUTP (Amersham Pharmacia Biotech), and 2 μl of Superscript (GibcoBRL) were added, and reaction was performed for 1 hour at 42° C. Again, 2 μl of Superscript (GibcoBRL) was added, and reaction was performed for 1 hour at 42° C. Then, 270 μl of TE buffer was added. The reaction solutions to which Cy3-dUTP and Cy5-dUTP, respectively, had been added, were combined and transferred to a single Microcon-30 (MILLIPORE). This was centrifuged at 10,000 rpm until the volume of liquid remaining in the upper cup of Microcon-30 was approximately 10 μl. The solution that passed the cup was transferred to a different tube. To the upper cup of Microcon-30, 500 μl of TE buffer and 15 μg of human Cot1 DNA (GibcoBRL) were added. This was centrifuged at 10,000 rpm until the volume of liquid remaining in the upper cup of Microcon-30 was approximately 10 μl. The upper cup of Microcon-30 was removed and was placed into a new centrifuge tube in an inverted manner. The solution was collected into the centrifuge tube by centrifugation at 3,000 rpm for 3 minutes. To the recovered solution, 10 μg of yeast tRNA (Sigma), 4 μg of poly (dA) (Sigma), and distilled water were added so that the total volume would be 8 μl. To this solution, 1.7 μl of 20X SSC and 0.3 μl of 10% SDS were added, and denaturation was performed for 1 minute at 100° C. By leaving the sample at room temperature for 30 minutes to slowly cool the sample, the cDNA sample containing labeled cDNAs was prepared.

(2) Measurement of expression levels using microarrays

The cDNA sample prepared in (1) was added dropwise to a microarray in which DNA fragments containing a portion or the whole of the genes and/or ESTs indicated in row “Gene Name” of Table 1 shown below were immobilized on a slide glass (Nisshinbo). The ten genes of NOs. 3, 53, 54, 79, 98, 116, 127, 128, 141, and 148 in Table 1 are genes constitutively expressing regardless of the presence or absence of estrogen, and the DNA fragments containing a portion or the whole of these genes were immobilized on the basal plate as control genes. In addition, changes in the expression levels of the ten control genes and the 138 kinds of genes and ESTs in the presence of 10 nM 17β-estradiol in MCF-7 cells are shown in row “BDE” of Table 1. Here, negative BDE values indicate decrease in expression levels.

After dropwise addition of the cDNA sample onto the DNA microarray, a cover glass (Matsunami) was placed. To prevent drying of the slide glass, the microarray was placed into a hybridization chamber and the chamber was sealed. The hybridization chamber was incubated at 65° C. for 10 to 20 hours. The slide glass was taken out of the hybridization chamber and soaked into 2X SSC/0.2X SDS solution to remove the cover glass. The slide glass was washed with gentle shaking in 1X SSC and was washed again with gentle shaking in 0.2X SSC. After the slide glass was dried, the amount of fluorescence on the DNA microarrays was measured using a DNA microarray fluorescence scanner (Virtek, ChipReader) The results are shown in row “E(example)” of Table 1 shown below. As to genes and ESTs excluding the control genes, the weighting factors (row “K” in Table 1) were set based on the respective characteristics. As to the ten control genes, K was taken to be zero.

| No. | Gene Name | GenBank ID | BDE | K | E (example) | BDE′ |
|---|---|---|---|---|---|---|
| 1 | absent in melanoma 1 | U83115 | −3.1 | 1.2 | −1.1 | −2.61 |
| 2 | aconitase 2, mitochondrial | U87939 | −2.3 | 0.5 | 1.0 | 2.37 |
| 3 | actin, beta | X00351 | −1.0 | 0.0 | 1.1 | 2.61 |
| 4 | activating transcription factor 3 | N39944 | 4.9 | 1.0 | 0.0 | 0 |
| 5 | alanyl-tRNA synthetase | D32050 | 3.5 | 0.5 | 1.9 | 4.5 |
| 6 | aldo-keto reductase family 1, member C4 | T73188 | 3.6 | 0.5 | 1.2 | 2.84 |
| 7 | alpha-1-antichymotrypsin | X00947 | 2.7 | 0.5 | 2.9 | 6.87 |
| 8 | amphiregulin | M30704 | 8.2 | 1.0 | 5.0 | 11.85 |
| 9 | antigen identified by monoclonal antibodies 4F2, TRA1.10, TROP4, and T43 | AB018010 | 5.2 | 0.5 | 2.6 | 6.16 |
| 10 | argininosuccinate synthetase | AI660571 | 2.9 | 0.5 | 2.7 | 6.4 |
| 11 | asparagine synthetase | L35946 | 12.1 | 0.5 | 4.2 | 9.95 |
| 12 | B-factor, properdin | X72875 | 3.2 | 0.5 | 1.0 | 2.37 |
| 13 | bassoon | Y18448 | −3.0 | 0.5 | 0.0 | 0 |
| 14 | cadherin 18 | U59325 | −3.7 | 1.0 | 1.1 | 2.61 |
| 15 | calpain, small polypeptide | AC002984 | −2.8 | 0.5 | −1.4 | −3.32 |
| 16 | carnitine palmitoyltransferase I, liver | L39211 | −2.6 | 1.0 | 1.2 | 2.84 |
| 17 | catenin, delta 2 | U96136 | −3.2 | 1.0 | −1.2 | −2.84 |
| 18 | cathepsin D | M63138 | 2.4 | 0.5 | 2.2 | 5.21 |
| 19 | CCAAT/enhancer binding protein (C/EBP), beta | AA557306 | 5.1 | 1.0 | 2.1 | 4.98 |
| 20 | CDC6 homolog | AF022109 | 2.0 | 1.0 | −1.1 | −2.61 |
| 21 | CDP-diacylglycerol-inositol 3-phosphatidyltransferase | AF014807 | −2.0 | 0.5 | −1.2 | −2.84 |
| 22 | ohromobox homolog 1 | AL046741 | −2.1 | 0.5 | −1.4 | −3.32 |
| 23 | clusterin | X14723 | −3.4 | 0.5 | −1.2 | −2.84 |
| 24 | corneodesmosin | L20815 | 2.2 | 0.5 | −1.1 | −2.61 |
| 25 | cyclin AI | U66838 | 3.4 | 1.0 | 0.0 | 0 |
| 26 | DKFZP566G223 protein | AI261366 | 3.0 | 0.5 | 2.6 | 6.16 |
| 27 | down-regulated in adenoma | L02785 | 4.0 | 1.2 | −1.1 | −2.61 |
| 28 | early growth response 3 | X63741 | 8.8 | 1.0 | 1.9 | 4.5 |
| 29 | EGF-containing fibulin-like extracellular matrix protein 1 | U03877 | −6.7 | 1.0 | −3.7 | −8.77 |
| 30 | enolase 2 | X51956 | −2.7 | 0.5 | −1.8 | −4.27 |
| 31 | enolase 3 | X56832 | −3.3 | 0.5 | −1.6 | −3.79 |
| 32 | estrogen receptor 1 | X03635 | −3.0 | 1.5 | −1.4 | −3.32 |
| 33 | ESTs | AA587912 | 6.3 | 0.5 | 1.3 | 3.08 |
| 34 | ESTs | AA629308 | 3.2 | 0.5 | 1.2 | 2.84 |
| 35 | ESTs | AI188401 | 2.2 | 0.5 | −1.2 | −2.84 |
| 36 | ESTs | AI332415 | 2.1 | 0.5 | −1.2 | −2.84 |
| 37 | ESTs | AI492976 | 2.1 | 0.5 | −1.1 | −2.61 |
| 38 | ESTs | AI949781 | 2.3 | 0.5 | 1.1 | 2.61 |
| 39 | ESTs | N35555 | 11.4 | 0.5 | 3.7 | 8.77 |
| 40 | ESTs | U82984 | 3.1 | 0.5 | 1.3 | 3.08 |
| 41 | ESTs | AA682502 | 3.1 | 0.5 | 2.0 | 4.74 |
| 42 | ESTs | AA975298 | 3.7 | 0.5 | 2.0 | 4.74 |
| 43 | ESTs | AI767533 | 2.0 | 0.5 | −1.1 | −2.61 |
| 44 | eukaryotic translation initiation factor 3, subunit 9 | U78525 | −2.2 | 0.5 | −1.2 | −2.84 |
| 45 | exportin | AF039022 | 3.0 | 0.5 | 1.8 | 4.27 |
| 46 | ferritin, heavy polypeptide 1 | AA102267 | 2.9 | 0.5 | 0.0 | 0 |
| 47 | FOS-like antigen 2 | X16706 | 5.1 | 1.0 | 1.8 | 4.27 |
| 48 | FOS-like antigen 2 | AI088306 | 3.1 | 1.0 | 1.5 | 3.55 |
| 49 | fructose-bisphosphatase 1 | M19922 | −2.6 | 0.5 | −1.6 | −3.79 |
| 50 | fucosyltransferase 8 | Y17977 | −3.2 | 0.5 | −1.6 | −3.79 |
| 51 | GATA-binding protein 3 | X55122 | −2.6 | 1.0 | −1.5 | −3.55 |
| 52 | general transcription factor II, i, pseudogene 1 | AI700706 | −2.8 | 1.0 | −1.7 | −4.03 |
| 53 | general transcription factor IIH, polypeptide 2 | AI239815 | −1.1 | 0.0 | 1.1 | 2.61 |
| 54 | glutamate-cysteine ligase | L35546 | −1.1 | 0.0 | 1.0 | 2.37 |
| 55 | glutamic-oxaloacetic transaminase 1 | M37400 | 3.9 | 0.5 | 1.5 | 3.55 |
| 56 | glutamine-fructose-6-phosphate transaminase 1 | M90516 | 3.2 | 0.5 | 1.1 | 2.61 |
| 57 | glycyl-tRNA synthetase | D30658 | 3.1 | 0.5 | 1.7 | 4.03 |
| 58 | granulin | AF055008 | −2.2 | 0.5 | −1.0 | −2.37 |
| 59 | H3 histone, family 3B | Z48950 | 2.8 | 1.0 | 1.4 | 3.32 |
| 60 | heat shock 70kD protein 1 | M59828 | −3.6 | 1.0 | −2.0 | −4.74 |
| 61 | heat shock 70kD protein 5 | AI878886 | 4.9 | 0.5 | 1.9 | 4.5 |
| 62 | histone deacetylase 6 | AJ011972 | 2.0 | 1.0 | −1.1 | −2.61 |
| 63 | Homo sapiens clone 23783 mRNA sequence | AF054996 | −2.0 | 0.5 | −1.2 | −2.84 |
| 64 | Homo sapiens DNA from chromosome 19, cosmid F21856 | AC004030 | −2.6 | 0.5 | −1.1 | −2.61 |
| 65 | HS1 binding protein | N28312 | 2.0 | 0.5 | 1.3 | 3.08 |
| 66 | Human clone 23855 mRNA, partial cds | U79302 | −2.1 | 0.5 | 1.0 | 2.37 |
| 67 | Human insulin-like growth factor binding protein 5 | L27560 | −12.8 | 1.0 | 1.1 | 2.61 |
| 68 | Human mRNA for KIAA0018 gene, complets cds | D13643 | −3.0 | 0.5 | −1.5 | −3.55 |
| 69 | hyaluronan-mediated motility receptor (RHAMM) | U29343 | 3.5 | 1.5 | 1.4 | 3.32 |
| 70 | Incyte EST | AI185199 | 3.8 | 0.5 | −1.1 | −2.61 |
| 71 | Incyte EST | AI880413 | 3.0 | 0.5 | 1.2 | 2.84 |
| 72 | Incyte EST | X625858 | 3.0 | 0.5 | 1.7 | 4.03 |
| 73 | inhibitor of DNA binding 4, dominant negative helix-loop-helix protein | AL022726 | 2.1 | 0.5 | −1.2 | −2.84 |
| 74 | insulin-like growth factor binding protein 5 | AA374325 | −7.0 | 1.0 | 0.0 | 0 |
| 75 | insulin-like growth factor binding protein 4 | M62403 | 3.5 | 1.0 | 0.0 | 0 |
| 76 | interferon stimulated gene (20kD) | X89773 | 4.0 | 0.5 | 1.1 | 2.61 |
| 77 | isocitrate dehydrogenase 2 (NADP+) | X69433 | −3.7 | 0.5 | −1.1 | −2.61 |
| 78 | isoleucine-tRNA synthetase | D28473 | 2.0 | 0.5 | 2.3 | 5.45 |
| 79 | keratin 6B | L42611 | 1.1 | 0.0 | −1.0 | −2.37 |
| 80 | keratin 8 | AI273887 | 2.2 | 0.5 | 1.3 | 3.08 |
| 81 | KIAA0058 | AL036958 | −2.1 | 0.5 | −1.3 | −3.08 |
| 82 | KIAA0123 | D21064 | −2.0 | 0.5 | −1.1 | −2.61 |
| 83 | KIAA0196 | D83780 | −2.2 | 0.5 | 1.0 | 2.37 |
| 84 | KIAA0307 | AB002305 | −3.6 | 0.5 | −1.8 | −4.27 |
| 85 | KIAA0551 | AB011123 | 2.0 | 0.5 | −1.2 | −2.84 |
| 86 | KIAA0587 | AB011159 | 2.4 | 0.5 | 1.5 | 3.55 |
| 87 | KIAA1051 | AB028974 | 6.4 | 0.5 | 1.7 | 4.03 |
| 88 | mal | X76220 | −2.0 | 0.5 | 1.0 | 2.37 |
| 89 | mannosidase, alpha, class 1A, member 1 | X74837 | 2.1 | 0.5 | 1.4 | 3.32 |
| 90 | matrix Gla protein | AA484893 | 8.2 | 0.5 | 2.3 | 5.45 |
| 91 | membrane component, chromosome 1, surface marker 1 | X13425 | −2.0 | 0.5 | −1.5 | −3.55 |
| 92 | methylene tetrahydrofolate dehydrogenase | X16396 | 3.9 | 0.5 | 2.4 | 5.69 |
| 93 | microtubule-associated protein 1B | L06237 | 3.9 | 0.5 | 2.1 | 4.98 |
| 94 | motilin | X15393 | 4.3 | 0.5 | 1.1 | 2.61 |
| 95 | MYC promoter-binding protein 1 | AA482422 | −2.6 | 1.2 | −1.5 | −3.55 |
| 96 | neuropeptide Y receptor Y1 | M84755 | 3.2 | 1.5 | 1.4 | 3.32 |
| 97 | nuclear receptor interacting protein 1 | X84373 | 3.0 | 1.5 | 2.3 | 5.45 |
| 98 | nucleophosmin | AA173870 | −1.1 | 0.0 | −1.1 | −2.61 |
| 99 | paired basic amino acid cleaving system 4 | D87993 | −3.5 | 0.5 | −1.2 | −2.84 |
| 100 | PDZ domain containing 1 | AF012281 | 3.4 | 0.5 | 2.5 | 5.92 |
| 101 | phorbol-12-myristate-13-acetate-induced protein 1 | D90070 | 4.2 | 1.2 | 1.1 | 2.61 |
| 102 | phosphoenolpyruvate carboxykinase 2 | X92720 | 7.6 | 0.5 | 2.1 | 4.98 |
| 103 | phosphoinositide-3-kinase, class 3 | Z46973 | 3.8 | 0.5 | −1.1 | −2.61 |
| 104 | pituitary tumor-transforming 1 | AA430241 | 2.5 | 1.2 | −1.1 | −2.61 |
| 105 | poliovirus receptor | X64116 | 2.0 | 1.5 | 1.1 | 2.61 |
| 106 | prostate differentiation factor | AA216685 | 5.0 | 1.0 | 1.4 | 3.32 |
| 107 | protein geranylgeranyltransferase type I, beta subunit | AA481712 | 2.0 | 0.5 | 1.0 | 2.37 |
| 108 | protein kinase C substrate 80K-H | J03075 | −2.2 | 0.5 | −1.0 | −2.37 |
| 109 | protein kinase C, delta | D10495 | −2.9 | 0.5 | −1.4 | −3.32 |
| 110 | protein tyrosine phosphatase, non-receptor type 18 | X79568 | −2.0 | 1.0 | 1.0 | 2.37 |
| 111 | quiescin Q6 | U97276 | −4.0 | 0.5 | −1.1 | −2.61 |
| 112 | ras homolog gene family, member C | L25081 | −2.1 | 1.2 | −1.6 | −3.79 |
| 113 | reticulocalbin 1, EF-hand calcium binding domain | D42073 | 2.0 | 0.5 | 1.7 | 4.03 |
| 114 | retinoblastoma-binding protein 8 | U72066 | 3.9 | 1.2 | 1.3 | 3.08 |
| 115 | Rho GDP dissociation inhibitor alpha | X69550 | −3.1 | 0.5 | −1.1 | −2.61 |
| 116 | ribosomal protein L35 | AI815757 | −1.0 | 0.0 | −1.2 | −2.84 |
| 117 | ribosomal protein S6 kinase, 90kD, polypeptide 3 | U08316 | 3.1 | 1.0 | 1.1 | 2.61 |
| 118 | S100 calcium-binding protein A9 (calgranulin B) | X06233 | 5.1 | 0.5 | 0.0 | 0 |
| 119 | S100 calcium-binding protein P | AI151190 | 4.7 | 0.5 | 1.6 | 3.79 |
| 120 | selenium binding protein 1 | U29091 | −4.4 | 0.5 | −2.1 | −4.98 |
| 121 | semaphorin | AI683760 | 3.6 | 0.5 | 1.4 | 3.32 |
| 122 | serine hydroxymethyltransferase 2 | U23143 | 2.2 | 0.5 | 1.8 | 4.27 |
| 123 | SH3-domain binding protein 5 (BTK-associated) | AB005047 | 2.7 | 0.5 | −1.0 | −2.37 |
| 124 | solute carrier family 1, member 4 | L14595 | 5.5 | 1.0 | 2.0 | 4.74 |
| 125 | solute carrier family 1, member 5 | AF105826 | 2.4 | 0.5 | 1.6 | 3.79 |
| 126 | solute carrier family 12, member 2 | U30246 | −4.3 | 1.0 | −1.6 | −3.79 |
| 127 | solute carrier family 29, member 2 | AI342303 | −1.1 | 0.0 | −1.1 | −2.61 |
| 128 | solute carrier family 5, member 1 | Z80998 | −1.0 | 0.0 | 1.2 | 2.84 |
| 129 | solute carrier family 7, member 5 | M80244 | 5.3 | 0.5 | 2.6 | 6.16 |
| 130 | sorbitol dehydrogenase | U07361 | −2.2 | 0.5 | −1.1 | −2.61 |
| 131 | stanniocalcin 2 | AB012664 | 5.5 | 0.5 | 1.0 | 2.37 |
| 132 | stearoyl-CoA desaturase | AA143530 | 2.0 | 0.5 | −1.1 | −2.61 |
| 133 | synaptogyrin 2 | AJ002308 | −2.3 | 1.0 | −1.2 | −2.84 |
| 134 | TATA box binding protein (TBP)-associated factor G | U21858 | 2.1 | 1.0 | 1.3 | 3.08 |
| 135 | tetraspan 1 | AF065388 | −3.8 | 0.5 | −1.2 | −2.84 |
| 136 | TGFB inducible early growth response | AF050110 | 3.4 | 1.0 | 1.2 | 2.84 |
| 137 | thrombospondin 3 | AI679881 | 2.0 | 0.5 | −1.1 | −2.61 |
| 138 | transcobalamin I | J05068 | 6.2 | 0.5 | 0.0 | 0 |
| 139 | trefoil factor 1 | X05030 | 11.7 | 1.5 | 4.9 | 11.61 |
| 140 | tryptophanyl-tRNA synthetase | X62570 | 5.7 | 0.5 | 2.3 | 5.45 |
| 141 | tumor necrosis factor receptor superfamily, member 7 | AA994925 | −1.1 | 0.0 | 1.1 | 2.61 |
| 142 | tumor protein D52-like 1 | U44427 | 8.1 | 1.2 | 2.1 | 4.98 |
| 143 | tumor rejection antigen (gp96) 1 | X15187 | 3.3 | 1.2 | 1.5 | 3.55 |
| 144 | tyrosyl-tRNA synthetase | U89436 | 2.9 | 0.5 | 2.1 | 4.98 |
| 145 | U5 snRNP-specific protein, 116 kD | D21163 | −2.0 | 0.5 | 1.0 | 2.37 |
| 146 | unc-51 (C. elegans)-like kinase 1 | AB018265 | 3.6 | 0.5 | 1.1 | 2.61 |
| 147 | v-jun avian sarcoma virus 17 oncogene homolog | AI598150 | 3.0 | 1.2 | −1.3 | −3.08 |
| 148 | zinc finger protein 147 | D21205 | −1.0 | 0.0 | −1.1 | −2.61 |

The evaluation value was calculated from the results shown in Table 1 according to

(evaluation value)=(ΣKn|BDE'n/BDEn|)/(ΣKn|BDEn/BDEn|)

=(ΣKn|BDE'n/BDEn|)/(ΣKn)

where (n=1 to 138), and the result was 64.09/96.00=0.668. That is, when the expression level of the genes and ESTs in MCF-7 cells in the presence of 10 nM 17β-estradiol was taken to be 1.00, the expression level in the cells evaluated in this example was 0.668. Thus, the microarray that has DNA fragments containing a portion or the whole of the genes and ESTs of Table 1 distinguished the effect of 17β-estradiol. As these results show, the effect of chemical substances having estrogen-like activity, such as environmental hormones, on cells can be evaluated by the above-mentioned microarray.

What is claimed is:

1. A microarray comprising a basal plate and a DNA fragment immobilized on the basal plate, the DNA fragment comprising a portion or the whole of a gene and/or an EST whose expression level is affected by a chemical substance having estrogen-like activity.

2. The microarray of claim 1, wherein the gene and/or the EST is selected from the group consisting of those whose expression level changes in a breast cancer-derived MCF-7 cell in the presence of estrogen.

3. The microarray of claim 1 or 2, wherein the gene and/or the EST belongs to any one of groups (1), (2), and (3) below:
group (1): a gene and/or an EST whose expression level increases owing to a chemical substance having estrogen-like activity,
group (2): a gene and/or an EST whose expression level decreases owing to a chemical substance having estrogen-like activity, and
group (3): a gene and/or an EST whose expression level changes smaller and whose expression level is higher, compared to the groups (1) and (2).

4. The microarray of claim 3, wherein at least one or more genes and/or ESTs selected from the group consisting of genes and/or ESTs belonging to the group (1), genes and/or ESTs belonging to the group (2), and genes and/or ESTs belonging to the group (3) are immobilized.

5. The microarray of any one of claims 1 to 4, further comprising a DNA fragment immobilized on the basal plate, the DNA fragment comprising a portion or the whole of a gene and/or an EST constitutively expressing regardless of the presence or absence of a chemical substance having estrogen-like activity.

6. The microarray of claim 1 or 2, wherein the gene and/or the EST belongs to any one of groups (4), (5), (6), (7), and (8) below:
group (4): a gene and/or an EST related to cancer,
group (5): a gene and/or an EST involved in cell proliferation and/or division,
group (6): a gene and/or an EST encoding a polypeptide functioning as a receptor,
group (7): a gene and/or an EST involved in transcription reaction, and
group (8): a gene and/or an EST involved in cell structure.

7. The microarray of claim 6, wherein at least one or more genes and/or ESTs selected from the group consisting of genes and/or ESTs belonging to the group (4), genes and/or ESTs belonging to the group (5), genes and/or ESTs belonging to the group (6), genes and/or ESTs belonging to the group (7), and genes and/or ESTs belonging to the group (8) are immobilized.

8. The microarray of claim 1 or 2, wherein the gene and/or the EST belongs to any one of groups (9), (10), and (11) below:
group (9): a gene and/or an EST functioning in a tissue on which estrogen acts,
group (10): a gene and/or an EST characteristic of an epithelial cell, and
group (11): a gene and/or an EST functioning in a nerve cell.

9. The microarray of claim 8, wherein at least one or more genes and/or ESTs selected from the group consisting of genes and/or ESTs belonging to the group (9), genes and/or ESTs belonging to the group (10), and genes and/or ESTs belonging to the group (11) are immobilized.

10. The microarray of claim 1 or 2, wherein the gene and/or the EST belongs to group (12) or (13) below:
group (12): a gene and/or an EST whose expression level increases owing to estrogen in a cell resistant to an antagonist of estrogen, and
group (13): a gene and/or an EST whose expression level decreases owing to estrogen in a cell resistant to an antagonist of estrogen.

11. The microarray of claim 10, wherein at least one or more genes and/or ESTs selected from the group consisting of genes and/or ESTs belonging to the group (12), and genes and/or ESTs belonging to the group (13) are immobilized.

12. A method for evaluating activity of a compound having estrogen-like activity, the method comprising utilizing the microarray of any one of claims 1 to 11.

13. A method for evaluating activity of a compound having estrogen-like activity, the method comprising the steps of:
(a) preparing a nucleic acid sample from a cell with which an effect of a compound having estrogen-like activity is to be evaluated,
(b) contacting the nucleic acid sample with the microarray of any one of claims 1 to 11,
(c) detecting a nucleic acid hybridizing with the microarray, and
(d) comparing a result of the detecting of (c) to a result detected using a nucleic acid sample prepared from a control cell.

14. The method of claim 13, wherein the control cell is an MCF-7 cell treated with 17β-estradiol.
